(19) United States
(12) Patent Application Publication
(10) Pub. No.: US 2022/0172844 A1
(43) Pub. Date: Jun. 2, 2022

(54) MACHINE LEARNING SYSTEM AND METHOD, INTEGRATION SERVER, INFORMATION PROCESSING APPARATUS, PROGRAM, AND INFERENCE MODEL CREATION METHOD

(71) Applicant: FUJIFILM Corporation, Tokyo (JP)

(72) Inventor: Daiki UEHARA, Tokyo (JP)

(73) Assignee: FUJIFILM Corporation, Tokyo (JP)

(21) Appl. No.: 17/673,764

(22) Filed: Feb. 16, 2022

Related U.S. Application Data
(63) Continuation of application No. PCT/JP2020/022609, filed on Jun. 9, 2020.

(30) Foreign Application Priority Data
Sep. 26, 2019 (JP) 2019-175714

(51) Int. Cl. G16H 50/20; G06N 3/08

(52) U.S. Cl. CPC G16H 50/20; G06N 3/08

(57) ABSTRACT

There are provided a machine learning system and method, an integration server, an information processing apparatus, a non-transitory computer readable medium storing a program, and an inference model creation method capable of ensuring a learning accuracy in federated learning. Each client terminal executes machine learning of the learning model using data stored in a medical institution, and transmits a learning result to the integration server. The integration server divides the plurality of client terminals into a plurality of client clusters, and creates master model candidates by integrating the learning results for each client cluster. The integration server evaluates an inference accuracy of each master model candidate, and in a case where a master model candidate having an inference accuracy lower than an accuracy threshold value is detected, extracts a client terminal as an accuracy deterioration cause from the client cluster used for creation of the master model candidate.

### CROSS-REFERENCE TO RELATED APPLICATIONS

This application is a Continuation of PCT International Application No. PCT/JP2020/022609 filed on Jun. 9, 2020, which claims priority under 35 U.S.C. § 119(a) to Japanese Patent Application No. 2019-175714 filed on Sep. 26, 2019. Each of the above application is hereby expressly incorporated by reference, in its entirety, into the present application.

### BACKGROUND OF THE INVENTION

### 1. Field of the Invention

The present invention relates to a machine learning system and method, an integration server, an information processing apparatus, a non-transitory computer readable medium storing a program, and an inference model creation method, and particularly relates to a machine learning technique using a federated learning mechanism.

### 2. Description of the Related Art

In development of medical artificial intelligence (AI) using deep learning, it is necessary to train an AI model. However, for this learning, it is necessary to extract learning data such as a diagnosis image from a medical institution to an external development site or to an external development server. For this reason, there are few medical institutions that can cooperate in providing learning data. Further, even in a case where learning data is provided from a medical institution, there is always a privacy-related risk.

On the other hand, in a case where a federated learning mechanism is used, the federated learning mechanism being proposed in H. Brendan, McMahan Eider, Moore Daniel Ramage, Seth Hampson, and Blaise Agüera y Arcas, “Communication-Efficient Learning of Deep Networks from Decentralized Data”, arXiv:1602.05629v3 [cs.LG], 28 Feb. 2017, learning is performed on a terminal in which data for training exists, and only a weight parameter of a network model that is a learning result on each terminal is transmitted from a terminal group to an integration server. That is, in federated learning, learning data is not provided to the integration server, and only data of the learning result on each terminal is provided from the terminal to the integration server.

For this reason, learning can be performed without extracting data that requires consideration for privacy to the outside. Thus, federated learning is a technique that has been attracting attention in recent years.

In Micah J. Sheller, G. Anthony Reina, Brandon Edwards, Jason Martin, and Spyridon Bakas, “Multi-Institutional Deep Learning Modeling Without Sharing Patient Data: A Feasibility Study on Brain Tumor Segmentation”, arXiv:1810.04304v2 [cs.LG], 22 Oct. 2018, a result of an example in which federated learning is applied to development of medical AI is reported.

### SUMMARY OF THE INVENTION

In a case where federated learning is used for development of medical AI, it is not necessary to extract data such as a diagnosis image. However, many medical institutions are involved in learning, and as a result, a mechanism to ensure a learning accuracy is required.

In Eugene Bagdasaryan, Andreas Veit, Yiqing Hua, Deborah Estrin, and Vitaly Shmatikov, “How to Backdoor Federated Learning”, arXiv:1807.00459v2 [cs.CR], 1 Oct. 2018, in a case where incorrect data is mixed in learning by some clients participating in federated learning, in terms of federated learning algorithms, it is pointed out that there is no effective way to prevent an accuracy decrease of a master model due to mixing of the incorrect data in learning. For example, in a case where some clients participating in learning intentionally input data including misrecognized image content and learning is performed, the master model may perform an incorrect determination on a specific image. It is said that there is currently no way to effectively prevent the problems in an algorithmic manner.

In addition, even though it is not intended, for example, in a case where misdiagnosed data is mixed in learning, the problems may occur. For example, in a case where a model is trained using data with which a plurality of misdiagnoses for a specific disease are performed by a medical institution, there is no mechanism to ensure accuracy.

As described in “Section 4.3” of Micah J. Sheller, G. Anthony Reina, Brandon Edwards, Jason Martin, and Spyridon Bakas, “Multi-Institutional Deep Learning Modeling Without Sharing Patient Data: A Feasibility Study on Brain Tumor Segmentation”, arXiv:1810.04304v2 [cs.LG], 22 Oct. 2018, in an existing federated learning mechanism structure alone, in a situation where an unspecified number of medical institutions participate in learning, in order to ensure a learning accuracy, it is necessary to cause each medical institution to perform learning using data prepared for learning.

One of the tasks to ensure a learning accuracy is to select a client which has a problem unsuitable for learning and which is an accuracy deterioration cause and a client which has no problem from a client group involved in learning. Further, one of the other tasks is to prevent a client which is unsuitable for learning from affecting learning.

The present invention has been made in view of such circumstances, and an object of the present invention is to solve at least one of the above multiple problems and to provide a machine learning system and method, an integration server, an information processing apparatus, a non-transitory computer readable medium storing a program, and an inference model creation method capable of preventing an accuracy decrease of a master model caused by some clients in a case where a federated learning mechanism for performing training of an AI model is adopted, without extracting personal information such as a diagnosis image that requires consideration for privacy from a medical institution.

According to an aspect of the present disclosure, there is provided a machine learning system including: a plurality of client terminals; and an integration server, wherein the integration server comprises a first processor and a non-transitory first computer-readable medium storing a trained master model and each of the plurality of client terminals comprises a second processor. The second processor executes machine learning of a learning model using, as learning data, data stored in a data storage apparatus of a medical institution and transmits a learning result of the learning model to the integration server. The first processor synchronizes the learning model of each client terminal with the master model before training of the learning model is performed on each of the plurality of client terminals, receives each of the learning results from the plurality of client terminals, creates a plurality of client clusters by dividing the plurality of client terminals into a plurality of groups, creates master model candidates for each of the client clusters by integrating the learning results for each of the client clusters, detects the master model candidate having an inference accuracy lower than an accuracy threshold value by evaluating the inference accuracy of each of the master model candidates created for each of the client clusters, and extracts a client terminal as an accuracy deterioration cause from the client cluster used for creation of the master model candidate having the inference accuracy lower than the accuracy threshold value.

According to this aspect, the plurality of client terminals participating in learning are divided into the plurality of client clusters, and a plurality of master model candidates are created by integrating the learning results for each client cluster. By evaluating the inference accuracy of each of the plurality of master model candidates, the low-accuracy master model candidate having the inference accuracy lower than the accuracy threshold value and the client cluster used for creation of the low-accuracy master model candidate (low-accuracy client cluster) are specified. It is considered that the low-accuracy client cluster includes at least one client terminal as an accuracy deterioration cause. The first processor extracts a client terminal as the accuracy deterioration cause by narrowing down the client terminals as the accuracy deterioration causes from the low-accuracy client cluster.

The first processor may extract only the client terminal as an actual accuracy deterioration cause, or may extract a combination of the client terminals suspected as the accuracy deterioration causes. That is, a part of the client terminals extracted by the first processor may include the client terminal that does not actually correspond to the accuracy deterioration cause.

According to this aspect, it is possible to find the client terminal that is not suitable for learning and that is an accuracy deterioration cause from the plurality of client terminals participating in learning. Thereby, it is possible to select a client terminal having a problem in learning accuracy and a client terminal having no problem in learning accuracy. Therefore, it is possible to perform various measures such as excluding the extracted client terminal as an accuracy deterioration cause from subsequent learning, relatively reducing a contribution rate (for example, a weight in integration) of the learning result obtained from the client terminal as the accuracy deterioration cause, or notifying the client terminal as the accuracy deterioration cause of a request for cause investigation and improvement. According to this aspect, it is possible to ensure the learning accuracy based on information of the client terminal as the accuracy deterioration cause, and it is possible to prevent an accuracy decrease of the master model caused by some clients.

The “plurality of client terminals” may be an unspecified large number of client terminals. The client terminal may be configured to include a “data storage apparatus of a medical institution”, or the “data storage apparatus of a medical institution” and the “client terminal” may be separate apparatuses.

In the machine learning system according to another aspect of the present disclosure, the first processor may exclude the client terminal as the accuracy deterioration cause from subsequent learning.

In the machine learning system according to still another aspect of the present disclosure, the first computer-readable medium may store information of the client terminal as the accuracy deterioration cause.

In the machine learning system according to still another aspect of the present disclosure, each of the plurality of client terminals may be a terminal provided in a medical institution network of different medical institutions.

In the machine learning system according to still another aspect of the present disclosure, the integration server may be provided in a medical institution network or outside the medical institution network.

In the machine learning system according to still another aspect of the present disclosure, the learning result transmitted from the client terminal to the integration server may include a weight parameter of the trained learning model.

In the machine learning system according to still another aspect of the present disclosure, the data used as the learning data may include at least one type of data among a two-dimensional image, a three-dimensional image, a moving image, time-series data, and document data.

In the machine learning system according to still another aspect of the present disclosure, each model of the learning model, the master model, and the master model candidate may be configured by using a neural network.

An appropriate network model is applied according to a type of the learning data and a type of data which is input in an inference.

In the machine learning system according to still another aspect of the present disclosure, the data used as the learning data may include a two-dimensional image, a three-dimensional image, or a moving image, and each model of the learning model, the master model, and the master model candidate may be configured by using a convolutional neural network.

In the machine learning system according to still another aspect of the present disclosure, the data used as the learning data may include time-series data or document data, and each model of the learning model, the master model, and the master model candidate may be configured by using a recursive neural network.

In the machine learning system according to still another aspect of the present disclosure, the number of the client terminals included in each of the plurality of client clusters may be the same, and the client terminal included in each client cluster may not be overlapped.

In the machine learning system according to still another aspect of the present disclosure, the first computer-readable medium may store information indicating a correspondence relationship as to which client cluster among the plurality of client clusters each of the plurality of master model candidates created is based on.

In the machine learning system according to still another aspect of the present disclosure, the first processor may determine whether or not the inference accuracy of the master model candidate is lower than the accuracy threshold value based on a comparison between an instantaneous value of the inference accuracy of each of the master model candidates and the accuracy threshold value, or based on a comparison between a statistical value of the inference accuracy in a learning iteration of each of the master model candidates and the accuracy threshold value.

The “statistical value” is a statistical value calculated by using a statistical algorithm, and may be a representative value such as an average value or a median value.

In a case where the master model candidate having the inference accuracy lower than the accuracy threshold value is detected, the first processor may notify information of the master model candidate related to the detection.

In the machine learning system according to still another aspect of the present disclosure, the integration server may further comprise a display device. The display device displays the inference accuracy in each learning iteration of each of the master model candidates created for each of the client clusters.

In the machine learning system according to still another aspect of the present disclosure, a verification data storage unit that stores verification data may be further comprised, and the first processor may evaluate the inference accuracy of the master model candidate using the verification data.

The verification data storage unit may be included in the integration server, or may be an external storage apparatus connected to the integration server.

According to still another aspect of the present disclosure, there is provided a machine learning method using a plurality of client terminals and an integration server, the method including: synchronizing a learning model of each client terminal with a trained master model stored in the integration server before training of the learning model is performed on each of the plurality of client terminals; executing machine learning of the learning model using, as learning data, data stored in a data storage apparatus of each of medical institutions different from each other by each of the plurality of client terminals; transmitting a learning result of the learning model to the integration server from each of the plurality of client terminals; receiving each of the learning results from the plurality of client terminals by the integration server; creating a plurality of client clusters by dividing the plurality of client terminals into a plurality of groups by the integration server; creating master model candidates for each of the client clusters by the integration server, by integrating the learning results for each of the client clusters; detecting the master model candidate having an inference accuracy lower than an accuracy threshold value by the integration server, by evaluating the inference accuracy of each of the master model candidates created for each of the client clusters; and extracting a client terminal as an accuracy deterioration cause from the client cluster used for creation of the master model candidate having the inference accuracy lower than the accuracy threshold value by the integration server.

According to still another aspect of the present disclosure, there is provided an integration server connected to a plurality of client terminals via a communication line, the server including: a first processor; and a first computer-readable medium as a non-transitory tangible medium in which a first program to be executed by the first processor is recorded. The first processor is configured to, according to an instruction of the first program, store a trained master model on the first computer-readable medium, synchronize a learning model of each client terminal with the master model before training of the learning model is performed on each of the plurality of client terminals, receive each of learning results from the plurality of client terminals, create a plurality of client clusters by dividing the plurality of client terminals into a plurality of groups, create master model candidates for each of the client clusters by integrating the learning results for each of the client clusters, detect the master model candidate having an inference accuracy lower than an accuracy threshold value by evaluating the inference accuracy of each of the master model candidates created for each of the client clusters, and extract a client terminal as an accuracy deterioration cause from the client cluster used for creation of the master model candidate having the inference accuracy lower than the accuracy threshold value.

According to still another aspect of the present disclosure, there is provided an information processing apparatus that is used as one of the plurality of client terminals connected to the integration server according to this aspect of the present disclosure via a communication line, the information processing apparatus including: a second processor; and a second computer-readable medium as a non-transitory tangible medium in which a second program to be executed by the second processor is recorded. The second processor is configured to, according to an instruction of the second program, execute machine learning of a learning model by setting, as the learning model in an initial state before learning is started, a learning model synchronized with the master model stored in the integration server and using, as learning data, data stored in a data storage apparatus of a medical institution, and transmit a learning result of the learning model to the integration server.

According to still another aspect of the present disclosure, there is provided a non-transitory computer readable medium storing a program causing a computer to function as one of the plurality of client terminals connected to the integration server according to this aspect of the present disclosure via a communication line, the program causing the computer to realize: a function of executing machine learning of a learning model by setting, as the learning model in an initial state before learning is started, a learning model synchronized with the master model stored in the integration server and using, as learning data, data stored in a data storage apparatus of a medical institution; and a function of transmitting a learning result of the learning model to the integration server.

According to still another aspect of the present disclosure, there is provided a non-transitory computer readable medium storing a program causing a computer to function as an integration server connected to a plurality of client terminals via a communication line, the program causing the computer to realize: a function of storing a trained master model; a function of synchronizing a learning model of each client terminal with the master model before training of the learning model is performed on each of the plurality of client terminals; a function of receiving each of learning results from the plurality of client terminals; a function of creating a plurality of client clusters by dividing the plurality of client terminals into a plurality of groups; a function of creating master model candidates for each of the client clusters by integrating the learning results for each of the client clusters; a function of detecting the master model candidate having an inference accuracy lower than an accuracy threshold value by evaluating the inference accuracy of each of the master model candidates created for each of the client clusters; and a function of extracting a client terminal as an accuracy deterioration cause from the client cluster used for creation of the master model candidate having the inference accuracy lower than the accuracy threshold value.

According to still another aspect of the present disclosure, there is provided an inference model creation method for creating an inference model by performing machine learning using a plurality of client terminals and an integration server, the method including: synchronizing a learning model of each client terminal with a trained master model stored in the integration server before training of the learning model is performed on each of the plurality of client terminals; executing machine learning of the learning model using, as learning data, data stored in a data storage apparatus of each of medical institutions different from each other by each of the plurality of client terminals; transmitting a learning result of the learning model to the integration server from each of the plurality of client terminals; receiving each of the learning results from the plurality of client terminals by the integration server; creating a plurality of client clusters by the integration server, by dividing the plurality of client terminals into a plurality of groups; creating master model candidates for each of the client clusters by the integration server, by integrating the learning results for each of the client clusters; detecting the master model candidate having an inference accuracy lower than an accuracy threshold value by the integration server, by evaluating the inference accuracy of each of the master model candidates created for each of the client clusters; extracting a client terminal as an accuracy deterioration cause from the client cluster used for creation of the master model candidate having the inference accuracy lower than the accuracy threshold value by the integration server; and creating an inference model having the inference accuracy higher than the inference accuracy of the master model based on the master model candidate having an inference accuracy equal to or higher than the accuracy threshold value by the integration server.

The inference model creation method is understood as an invention of a method of generating an inference model. The term “inference” includes concepts of prediction, estimation, classification, and determination. The inference model may also be called an “AI model”.

According to the present invention, it is possible to extract a client terminal that is not suitable for learning and that is an accuracy deterioration cause from the plurality of client terminals. Thereby, it is possible to prevent an accuracy decrease of the master model caused by some unsuitable client terminals.

### BRIEF DESCRIPTION OF THE DRAWINGS

FIG. 1 is a conceptual diagram illustrating an outline of a machine learning system according to an embodiment of the present invention.

FIG. 2 is a diagram schematically illustrating a system configuration example of the machine learning system according to the embodiment of the present invention.

FIG. 3 is a block diagram illustrating a configuration example of an integration server.

FIG. 4 is a block diagram illustrating a configuration example of a computer aided detection/diagnosis (CAD) server as an example of a client.

FIG. 5 is a flowchart illustrating an example of an operation of a client terminal based on a local learning management program.

FIG. 6 is a flowchart illustrating an example of an operation of the integration server based on a learning client selection program.

FIG. 7 is a flowchart illustrating an example of an operation of the integration server based on an evaluation program.

FIG. 8 is a block diagram illustrating an example of a hardware configuration of a computer.

### DESCRIPTION OF THE PREFERRED EMBODIMENTS

Hereinafter, preferred embodiments of the present invention will be described with reference to the accompanying drawings.

<<Outline of Machine Learning System>>

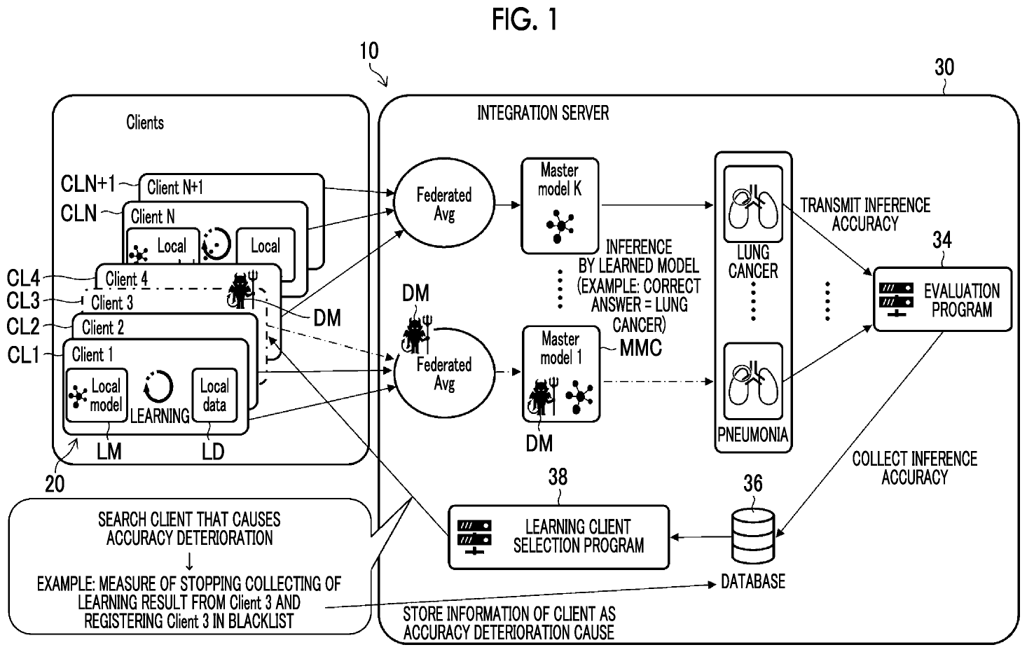

FIG. 1 is a conceptual diagram illustrating an outline of a machine learning system according to an embodiment of the present invention. A machine learning system 10 is a computer system that performs machine learning using a federated learning mechanism. The machine learning system 10 includes a plurality of clients 20 and an integration server 30. The federated learning is sometimes referred to as “federation learning”, “cooperative learning”, or “combination learning”.

Each of the plurality of clients 20 illustrated in FIG. 1 indicates a terminal in a medical institution that is provided on a network in a medical institution such as a hospital. Here, the “terminal” refers to a computing resource existing in a network that can safely access data in a medical institution, and the terminal may not physically exist in the medical institution. The client 20 is an example of a “client terminal” according to the present disclosure. A computer network in a medical institution is called a “medical institution network”.

It is assumed that each client 20 exists for each data group for training of an AI model. The term “for each data group” described herein may be understood as “for each medical institution” that includes a data group to be used for training the AI model. That is, it is assumed that one client exists for one medical institution.

In order to distinguish and display each of the plurality of clients 20, in FIG. 1 and subsequent drawings, representations such as “Client 1” and “Client 2” are used. A number after “Client” is an index as an identification number for identifying each client 20. In the present specification, the client 20 having an index of m is represented by “client CLm”. For example, the client CL1 represents “Client 1” in FIG. 1. m corresponds to a client identification number (ID number). Assuming that a total number of the clients 20 managed by the integration server 30 is M, m represents an integer equal to or larger than 1 and equal to or smaller than M. In FIG. 1, the clients 20 having indexes from m=1 to m=N+1 are illustrated. N represents an integer equal to or larger than 2. A set of the clients 20 of which the total number is M and which participate in learning is called a “learning client group” or a “population” of the clients 20.

Each client 20 stores local data LD in a local client storage apparatus. The local data LD is a data group accumulated by a medical institution to which the client 20 belongs.

Each client 20 includes a local learning management program as a distribution learning client program. Each client 20 performs an iteration for training a local model LM using the local data LD of the local client according to the local learning management program.

The local model LM is, for example, an AI model for medical image diagnosis that is incorporated in a CAD system. The term “CAD” includes concepts of both computer aided detection (CADe) and computer aided diagnosis (CADx). The local model LM is configured using, for example, a hierarchical multi-layer neural network. In the local model LM, network weight parameters are updated by deep learning using the local data LD as learning data. The weight parameters include a filter coefficient (a weight of a connection between nodes) of a filter used for processing of each layer and a bias of a node. The local model LM is an example of a “learning model for the client terminal” according to the present disclosure.

The “neural network” is a mathematical model for information processing that simulates a mechanism of a brain-nervous system. Processing using the neural network can be realized by using a computer. A processing unit including the neural network may be configured as a program module.

As a network structure of the neural network used for learning, an appropriate network structure is adopted according to a type of data used for input. The AI model for medical image diagnosis may be configured using, for example, various convolutional neural networks (CNNs) having a convolutional layer. The AI model that handles time-series data, document data, or the like may be configured using, for example, various recurrent neural networks (RNNs).

The plurality of clients 20 are connected to the integration server 30 via a communication network. In FIG. 1, a display of a devil mark DM indicates an occurrence of a deterioration in an inference accuracy of the AI model. The integration server 30 acquires learning results from each of the plurality of clients 20, and performs processing of creating a plurality of master model candidates MMC based on the learning results, processing of evaluating an inference accuracy of each master model candidate, and processing of selecting the client as an accuracy deterioration cause.

A location of the integration server 30 may exist on a computer network on which an entity developing the AI model has access rights, and a form of the server may be a physical server, a virtual server, or the like. The integration server 30 may be provided in a medical institution network, or may be provided outside a medical institution network. For example, the integration server 30 may be provided in a company that is located geographically away from a medical institution and that develops medical AI, or may be provided on a cloud.

The integration server 30 divides the plurality of clients 20 into K groups of client clusters, and creates K master model candidates MMC by integrating the learning results for each client cluster. K is an integer equal to or larger than 2.

The client cluster is a partial client group of the learning client group. The number of the clients 20 (the number of the clients) included in each of the K groups of the client clusters is the same. It is assumed that there is no overlap (non-overlapping) in the clients 20 included in each client cluster among different client clusters. Assuming that the number of the clients in the client cluster is Q, Q is an integer equal to or larger than 2. FIG. 1 illustrates an example of Q=3.

In FIG. 1, the clients CL1, CL2, and CL3 belong to the same client cluster, and the clients CL4, CLN, and CLN+1 belong to the same client cluster.

In FIG. 1, an arrow extending from a left side of a circle surrounding a display “Federated Avg” indicates that data of the trained local model LM is transmitted from each client 20 belonging to the same client cluster. The data of the local model LM as a learning result provided from each client 20 to the integration server 30 may be a weight parameter of the trained local model LM.

The circle surrounding the display “Federated Avg” represents processing of integrating the learning results. In the processing, the weights transmitted from each client 20 are integrated by averaging or the like, and a master model candidate MMC as an integration model is created. A method of integration processing is not limited to simple addition averaging. The weights may be weighted and integrated based on factors such as an attribute of the client 20, a past integration result, the number of pieces of data for each medical institution used for re-learning, and a level of a medical institution evaluated by a human.

In FIG. 1, “Master model 1” and “Master model K” illustrated at ends of arrows extending to a right side of the circle surrounding the display “Federated Avg” indicate the master model candidates MMC created from each client cluster. Assuming that an index for identifying each of the K groups of the client clusters is k, k represents an integer equal to or larger than 1 and equal to or smaller than K. In the present specification, the master model candidate MMC created by integrating the learning results of each client 20 belonging to the client cluster having an index k may be represented as “MMCk”. For example, in FIG. 1, the master model candidate MMC1 represents “Master model 1”.

The integration server 30 evaluates an inference accuracy of each master model candidate MMC using verification data prepared in advance. The verification data may be stored in an internal storage apparatus of the integration server 30, or may be stored in an external storage apparatus connected to the integration server 30.

The integration server 30 includes an evaluation program 34, a database 36, and a learning client selection program 38.

The evaluation program 34 collects an inference accuracy of each master model candidate MMC, and stores the inference accuracy in a data storage unit such as a database 36. The data storage unit may be a storage area of a storage apparatus in the integration server 30, or may be a storage area of an external storage apparatus connected to the integration server 30.

The evaluation program 34 compares the collected inference accuracy with an accuracy threshold value, and in a case where the inference accuracy of the master model candidate MMC is lower than the accuracy threshold value, notifies the learning client selection program 38 of the information.

In a case where a notification indicating that a master model candidate MMC having an inference accuracy lower than the accuracy threshold value exists is received from the evaluation program 34, the learning client selection program 38 performs processing of extracting a client as an inference accuracy deterioration cause.

The learning client selection program 38 searches for a client that causes an accuracy deterioration from the learning client group, and performs a measure of stopping collection of the learning results from the client as an accuracy deterioration cause and registering the corresponding client in a blacklist. The learning client selection program 38 stores information of the client as an accuracy deterioration cause in the data storage unit such as the database 36.

FIG. 1 illustrates an example in which the client CL3 is a client as an accuracy deterioration cause. The example illustrated in FIG. 1 is an example in which, assuming that the local model LM and the master model candidate MMC are CAD AI models for chest CT images, an inference is performed by inputting verification data in which a correct answer is “lung cancer” to the master model candidate MMC1 created by integrating the learning results of the client cluster including the client CL3 and an inference result represents an erroneous determination indicating “pneumonia”. Further, FIG. 1 illustrates an example in which an inference is similarly performed by inputting verification data in which a correct answer is “lung cancer” to the master model candidate MMCK created by integrating the learning results of the other client cluster and an inference result represents a correct determination indicating “lung cancer”.

<<Outline of Machine Learning Method>>

An example of a machine learning method by the machine learning system 10 according to the embodiment of the present invention will be described. The machine learning system 10 operates according to a procedure 1 to a procedure 11 to be described below.

[Procedure 1] As illustrated in FIG. 1, a distribution learning client program is executed on a terminal (client 20) in a medical institution that is provided on a computer network of the medical institution in which a data group for training of an AI model exists.

[Procedure 2] The integration server 30 synchronizes a latest version of the master model to be used for learning with the local model LM on each client 20 before each of the plurality of clients 20 starts learning. The master model is a trained AI model.

[Procedure 3] After synchronization with the latest version of the master model is performed, each client 20 performs learning on each terminal using the local data LD existing in the medical institution, and performs learning processing by the designated number of iterations. The local data LD used as the learning data may be, for example, a medical image and information associated with the medical image. The “associated information” may include information corresponding to a training signal. The number of iterations may be a fixed value, and more preferably, iterations of learning are performed until a stage where the inference accuracy is improved to be equal to or higher than a designated percentage.

[Procedure 4] After learning is completed, each client 20 transmits the learning result to the integration server 30. The learning result transmitted from the client 20 to the integration server 30 may be a weight parameter of the trained local model LM. The data of the weight parameter after training that is transmitted from the client 20 to the integration server 30 may be a difference from the weight parameter of the latest version of the master model synchronized with the integration server 30.

An attached document of a medical apparatus as the client 20 that uses the function according to the present embodiment describes that learning is performed as background processing within a range in which the learning does not interfere with medical work. In addition, the attached document describes that learning data to be used is data in the medical institution, that data to be transmitted to the outside is only a trained weight parameter, and that data by which an individual is identified is not transmitted.

[Procedure 5] The learning client selection program 38 which operates on the integration server 30 creates the master model candidates MMC for each client cluster by dividing the learning results transmitted from each client 20 into a plurality of client clusters and integrating the learning results. At this time, the learning client selection program 38 randomly extracts the clients 20 from the population without overlapping such that the number of clients included in each client cluster is the same, and stores information indicating which master model candidate MMC is created from which client cluster in the data storage unit such as the database 36. The database 36 is an example of an “association information storage unit” according to the present disclosure.

[Procedure 6] The evaluation program 34 performs a verification of the inference accuracy of each created master model candidate MMC. The verification of the inference accuracy is performed on the verification data. That is, the evaluation program 34 causes each master model candidate MMC to perform an inference by using, as an input, the verification data existing in the integration server 30, calculates an inference accuracy by comparing an inference result with correct answer data, and stores the inference accuracy of each master model candidate MMC in the data storage unit such as the database 36.

[Procedure 7] The evaluation program 34 compares the inference accuracy of each master model candidate MMC with the accuracy threshold value, and in a case where the inference accuracy of a certain master model candidate MMC is lower than the accuracy threshold value, notifies the learning client selection program 38 which master model candidate MMC is a model having the inference accuracy lower than the accuracy threshold value. When evaluating the inference accuracy, the inference accuracy of the master model candidate MMC that is to be compared with the accuracy threshold value may be an instantaneous value. However, in order to reduce a noise due to an abnormal value or the like, more preferably, a statistical value such as an average value or a median value of the inference accuracy may be used.

In the evaluation processing using the verification data, in a case where it is necessary to confirm a change in the inference accuracy of each master model candidate MMC, the change may be confirmed by using a display device (not illustrated) at this stage. The display device may be connected to the integration server 30.

[Procedure 8] In a case where a notification indicating that the master model candidate MMC having the inference accuracy lower than the accuracy threshold value exists is received, the learning client selection program 38 searches for, from the data storage unit such as the database 36, the client cluster that provides the learning result used for creating the master model candidate MMC having the inference accuracy lower than the accuracy threshold value.

[Procedure 9] Further, the learning client selection program 38 performs processing of extracting the client that particularly causes an accuracy deterioration, from the client clusters involved in creation of the master model candidate MMC having the inference accuracy lower than the accuracy threshold value. As an example of an extraction method used at this time, there is a method of calculating the inference accuracy of the local model LM of each client 20 in the client cluster involved in creation of the master model candidate MMC having a lower inference accuracy and extracting, as an accuracy deterioration cause, the client having a lower inference accuracy. Further, as an example of another extraction method, there is a method of mixing the local model LM of each client 20 in the client cluster involved in creation of the master model candidate MMC having a lower inference accuracy into the client cluster of the master model candidate MMC of which the inference accuracy is not lower than the accuracy threshold value and extracting, in a case where the inference accuracy of the master model candidate MMC as a mixing target is significantly deteriorated, the mixed client 20 as an accuracy deterioration cause.

[Procedure 10] The learning client selection program 38 stores, in the data storage unit such as the database 36, information of the client as an accuracy deterioration cause such that the client 20 which is an accuracy deterioration cause and which is extracted in the procedure 9 is not used for subsequent learning. The database 36 is an example of an “information storage unit” according to the present disclosure.

[Procedure 11] Thereafter, operations from the procedure 2 to the procedure 10 are repeated until a model having an inference accuracy higher than an inference accuracy for commercialization is obtained from the master model candidates MMC.

Thereby, a learning accuracy can be guaranteed, and it is possible to create an inference model having an inference accuracy equal to or higher than the accuracy threshold value. The machine learning method using the machine learning system 10 according to the present embodiment is understood as a method of creating an inference model.

<<System Configuration Example>>

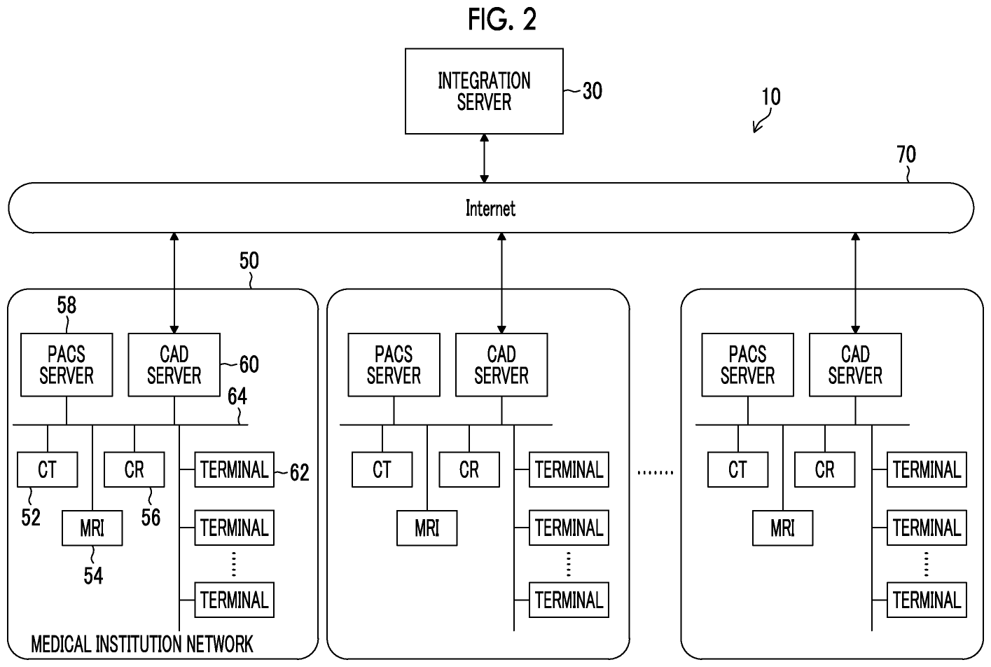

Next, an example of a specific configuration of the machine learning system 10 will be described. FIG. 2 is a diagram schematically illustrating a system configuration example of the machine learning system 10 according to the embodiment of the present invention. First, an example of a medical institution network 50 will be described. For simplicity of illustration, FIG. 2 illustrates an example in which the medical institution network 50 having the same system configuration is provided in each of a plurality of medical institutions. However, a medical institution network having a different system configuration for each medical institution may be provided.

The medical institution network 50 is a computer network including a computed tomography (CT) apparatus 52, a magnetic resonance imaging (MM) apparatus 54, a computed radiography (CR) apparatus 56, a picture archiving and communication systems (PACS) server 58, a CAD server 60, a terminal 62, and an internal communication line 64.

The medical institution network 50 is not limited to the CT apparatus 52, the MM apparatus 54, and the CR apparatus 56 illustrated in FIG. 2. Instead of some or all of the apparatuses or in addition to the apparatuses, at least one or a combination of a digital X-ray imaging apparatus, an angiography X-ray diagnosis apparatus, an ultrasound diagnosis apparatus, a positron emission tomography (PET) apparatus, an endoscopic apparatus, a mammography apparatus, and various inspection apparatuses (modalities) which are not illustrated may be included. There may be various combinations of types of test apparatuses connected to the medical institution network 50 for each medical institution.

The PACS server 58 is a computer that stores and manages various data, and includes a large-capacity external storage apparatus and database management software. The PACS server 58 performs a communication with another apparatus via the internal communication line 64, and transmits and receives various data including image data. The PACS server 58 receives various data including image data and the like generated by each inspection apparatus such as the CT apparatus 52, the MRI apparatus 54, and the CR apparatus 56 via the internal communication line 64, and stores and manages the data in a recording medium such as a large-capacity external storage apparatus.

A storage format of the image data and a communication between the apparatuses via the internal communication line 64 are based on a protocol such as digital imaging and communication in medicine (DICOM). The PACS server 58 may be a DICOM server that operates according to a DICOM specification. The data stored in the PACS server 58 can be used as learning data. The learning data created based on the data stored in the PACS server 58 may be stored in the CAD server 60. The PACS server 58 is an example of a “data storage apparatus of a medical institution” according to the present disclosure. Further, the CAD server 60 may function as the “data storage apparatus of a medical institution” according to the present disclosure.

The CAD server 60 corresponds to the client 20 described in FIG. 1. The CAD server 60 has a communication function for a communication with the integration server 30, and is connected to the integration server 30 via a wide area communication line 70. The CAD server 60 can acquire data from the PACS server 58 or the like via the internal communication line 64. The CAD server 60 includes a local learning management program for executing training of the local model LM on the CAD server 60 using the data group stored in the PACS server 58. The CAD server 60 is an example of a “client terminal” according to the present disclosure.

Various data stored in the database of the PACS server 58 and various information including the inference result by the CAD server 60 can be displayed on the terminal 62 connected to the internal communication line 64.

The terminal 62 may be a display terminal called a PACS viewer or a DICOM viewer. A plurality of terminals 62 may be connected to the medical institution network 50. A type of the terminal 62 is not particularly limited, and may be a personal computer, a workstation, a tablet terminal, or the like.

As illustrated in FIG. 2, a medical institution network having the same system configuration is provided in each of a plurality of medical institutions. The integration server 30 performs a communication with a plurality of CAD servers 60 via the wide area communication line 70. The wide area communication line 70 is an example of a “communication line” according to the present disclosure.

<<Configuration Example of Integration Server 30>>

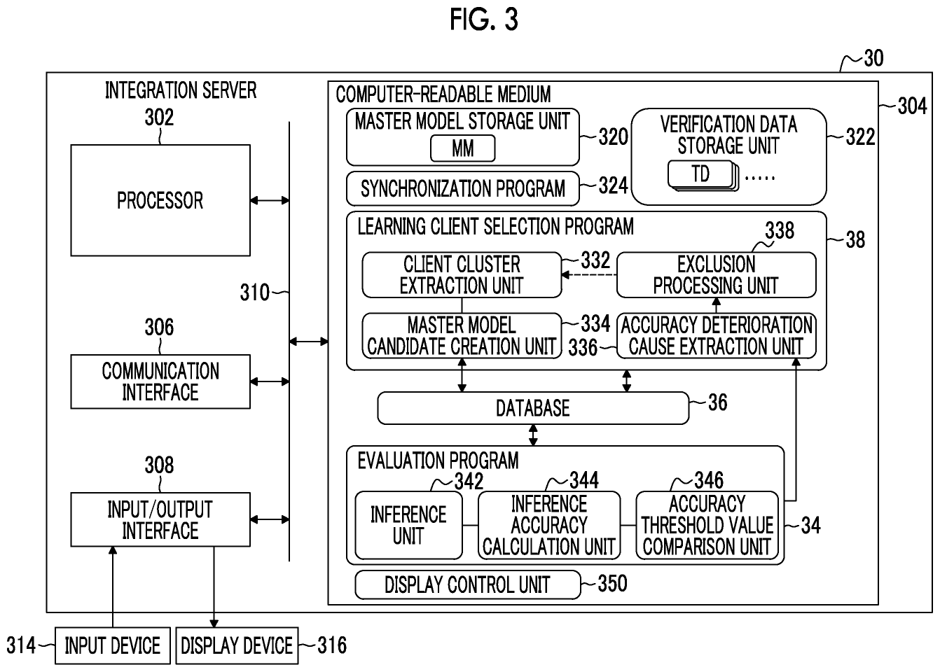

FIG. 3 is a block diagram illustrating a configuration example of the integration server 30. The integration server 30 can be realized by a computer system configured by using one or a plurality of computers. The integration server 30 is realized by installing and executing a program on a computer.

The integration server 30 includes a processor 302, a non-transitory tangible computer-readable medium 304, a communication interface 306, an input/output interface 308, a bus 310, an input device 314, and a display device 316. The processor 302 is an example of a “first processor” according to the present disclosure. The computer-readable medium 304 is an example of a “first computer-readable medium” according to the present disclosure.

The processor 302 includes a central processing unit (CPU). The processor 302 may include a graphics processing unit (GPU). The processor 302 is connected to the computer-readable medium 304, the communication interface 306, and the input/output interface 308 via the bus 310. The input device 314 and the display device 316 are connected to the bus 310 via the input/output interface 308.

The computer-readable medium 304 includes a memory as a main storage device and a storage as an auxiliary storage device. The computer-readable medium 304 may be, for example, a semiconductor memory, a hard disk drive (HDD) device, a solid state drive (SSD) device, or a combination of these devices.

The integration server 30 is connected to the wide area communication line 70 (refer to FIG. 2) via the communication interface 306.

The computer-readable medium 304 includes a master model storage unit 320, a verification data storage unit 322, and a database 36. The master model storage unit 320 stores data of a latest version of a master model MM. The verification data storage unit 322 stores a plurality of pieces of verification data TD which are used when verifying the inference accuracy of the integration model created by a master model candidate creation unit 334. The verification data TD is data in which input data and correct answer data are combined, and is also called test data. The verification data TD may be, for example, data provided by a university and the like.

The computer-readable medium 304 stores various programs, which include a synchronization program 324, the learning client selection program 38, and the evaluation program 34, and data. The synchronization program 324 is a program for providing the data of the master model MM to each client 20 via the communication interface 306 and synchronizing each local model LM with the master model MM. In a case where the processor 302 executes an instruction of the synchronization program 324, the computer functions as a synchronization processing unit. The synchronization program 324 may be incorporated as a program module of the learning client selection program 38.

In a case where the processor 302 executes an instruction of the learning client selection program 38, the computer functions as a client cluster extraction unit 332, a master model candidate creation unit 334, an accuracy deterioration cause extraction unit 336, and an exclusion processing unit 338. Further, in a case where the processor 302 executes an instruction of the evaluation program 34, the computer functions as an inference unit 342, an inference accuracy calculation unit 344, and an accuracy threshold value comparison unit 346.

The client cluster extraction unit 332 divides the learning results of the plurality of clients 20 received via the communication interface 306 into a plurality of client clusters having the same number of the clients. The communication interface 306 is an example of a “reception unit” according to the present disclosure. The client cluster extraction unit 332 is an example of a “client cluster creation unit” according to the present disclosure. The client cluster extraction unit 332 stores, in the database 36, information indicating a correspondence relationship between the information of the clients 20 belonging to each client cluster and the master model candidates MMC created for each client cluster.

The master model candidate creation unit 334 creates a plurality of master model candidates MMC by integrating the learning results for each client cluster. Information indicating a correspondence relationship as to which client cluster each master model candidate MMC created is based on is stored in the database 36.

The inference unit 342 executes an inference by each master model candidate MMC by inputting the verification data TD to each master model candidate MMC. The inference accuracy calculation unit 344 calculates an inference accuracy of each master model candidate MMC by comparing the inference result of each master model candidate MMC obtained from the inference unit 342 with the correct answer data. For example, as the correct answer data, data in which the number of lesions and correct clinical findings are added to the image data is used. The inference accuracy calculation unit 344 performs an accuracy verification a plurality of times through comparison with the verification data. The inference accuracy calculation unit 344 may calculate an accuracy average value of the master model candidate from the result obtained by performing the accuracy verification a plurality of times, and evaluate the accuracy average value as the inference accuracy of the master model candidate. The inference accuracy calculated by the inference accuracy calculation unit 344 is stored in the database 36.

The accuracy threshold value comparison unit 346 compares the inference accuracy of each master model candidate MMC with a predetermined accuracy threshold value, and determines whether or not a master model candidate MMC having an inference accuracy lower than the accuracy threshold value exists. In a case where a master model candidate MMC having an inference accuracy lower than the accuracy threshold value exists, the information is transmitted to the accuracy deterioration cause extraction unit 336. The inference unit 342, the inference accuracy calculation unit 344, and the accuracy threshold value comparison unit 346, which are realized by the evaluation program 34, are examples of an “accuracy evaluation unit” according to the present disclosure.

The accuracy deterioration cause extraction unit 336 performs processing of extracting a client that particularly causes an accuracy deterioration from the client cluster involved in the creation of the master model candidate MMC having an inference accuracy lower than the accuracy threshold value, and stores, in the database 36, information of the extracted client as an accuracy deterioration cause.

The client cluster involved in the creation of the master model candidate MMC having an inference accuracy lower than the accuracy threshold value is called a “low-accuracy client cluster”. The accuracy deterioration cause extraction unit 336 further divides the low-accuracy client cluster into, for example, a plurality of sub-clusters, mixes the client of the sub-cluster into the client cluster used in the creation of other master model candidates having no problem in the accuracy, and determines whether or not the accuracy is decreased in a case where the master model candidate is created. Thereby, in a case where a model of which the accuracy is significantly deteriorated compared to previous learning exists, it is determined that a client as an accuracy deterioration cause exists in the sub-cluster which is mixed into the model of which the accuracy is deteriorated. In a case where the number of the clients in the sub-cluster is relatively large, the accuracy deterioration cause extraction unit 336 further divides the sub-cluster including the client as an accuracy deterioration cause and repeats the same processing as described above. The accuracy deterioration cause extraction unit 336 repeats the iterations until the number of the clients in the client group (low-accuracy sub-cluster) including the client as an accuracy deterioration cause is sufficiently reduced. The client included in each sub-cluster among the plurality of sub-clusters may be overlapped.

As a result of the repetition, in a stage where the number of the clients in the client group including the client as an accuracy deterioration cause is sufficiently reduced, the accuracy deterioration cause extraction unit 336 registers, in the database 36, the client as an accuracy deterioration cause such that the client as an accuracy deterioration cause is not used in subsequent learning.

The exclusion processing unit 338 performs processing of excluding the corresponding client such that the client as an accuracy deterioration cause extracted by the accuracy deterioration cause extraction unit 336 is not used in subsequent learning. The “exclusion processing” may be, for example, at least one of not executing local learning processing on the client terminal as an accuracy deterioration cause, stopping of reception of the learning result from the client as an accuracy deterioration cause, or not adding the learning result received from the client as an accuracy deterioration cause to the integration processing.

The synchronization program 324, the learning client selection program 38, and the evaluation program 34 are examples of a “first program” according to the present disclosure.

Further, in a case where the processor 302 executes an instruction of a display control program, the computer functions as a display control unit 350. The display control unit 350 generates a display signal required for display output to the display device 316, and performs a display control of the display device 316.

The display device 316 is configured with, for example, a liquid crystal display, an organic electro-luminescence (OEL) display, a projector, or an appropriate combination thereof. The input device 314 is configured with, for example, a keyboard, a mouse, a touch panel, another pointing device, a voice input device, or an appropriate combination thereof. The input device 314 receives various inputs from an operator. The display device 316 and the input device 314 may be integrally configured by using a touch panel.

The display device 316 can display the inference accuracy in each learning iteration of each of the plurality of master model candidates MMC.

<<Configuration Example of CAD Server 60>>

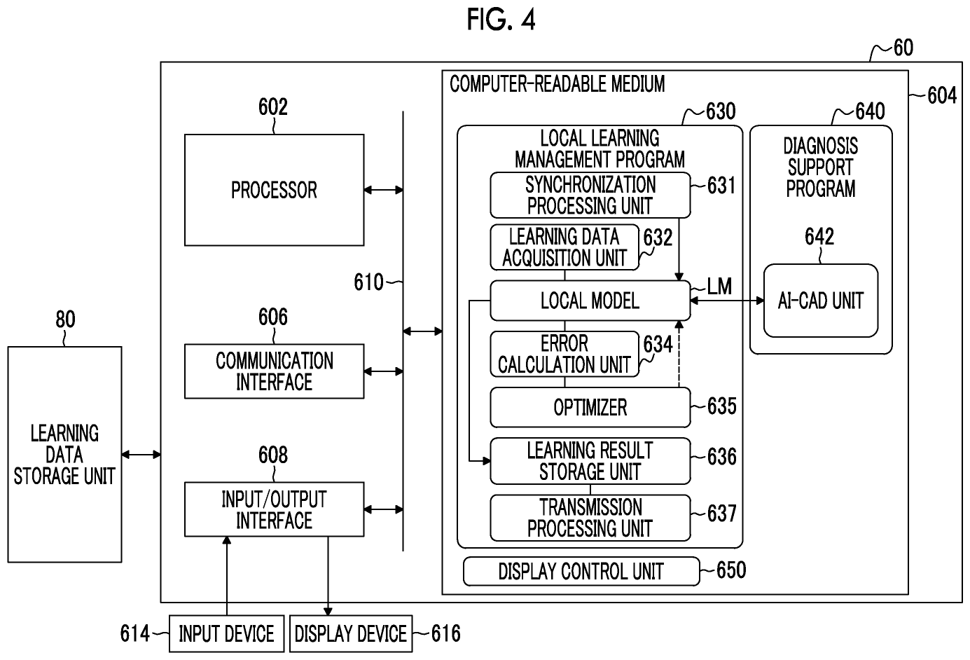

FIG. 4 is a block diagram illustrating a configuration example of the CAD server 60 as an example of the client 20. The CAD server 60 can be realized by a computer system configured by using one or a plurality of computers. The CAD server 60 is realized by installing and executing a program on a computer.

The CAD server 60 includes a processor 602, a non-transitory tangible computer-readable medium 604, a communication interface 606, an input/output interface 608, a bus 610, an input device 614, and a display device 616. The hardware configuration of the CAD server 60 may be the same as the hardware configuration of the integration server 30 described with reference to FIG. 3. That is, the hardware configuration of each of the processor 602, the computer-readable medium 604, the communication interface 606, the input/output interface 608, the bus 610, the input device 614, and the display device 616 in FIG. 4 is the same as the hardware configuration of each of the processor 302, the computer-readable medium 304, the communication interface 306, the input/output interface 308, the bus 310, the input device 314, and the display device 316 in FIG. 3.

The CAD server 60 is an example of an “information processing apparatus” according to the present disclosure. The processor 602 is an example of a “second processor” according to the present disclosure. The computer-readable medium 604 is an example of a “second computer-readable medium” according to the present disclosure.

The CAD server 60 is connected to a learning data storage unit 80 via the communication interface 606 or the input/output interface 608. The learning data storage unit 80 is configured to include a storage that stores learning data to be used for machine learning by the CAD server 60. The “learning data” is training data used for machine learning, and is synonymous with “data for learning” or “training data”. The learning data stored in the learning data storage unit 80 is the local data LD described with reference to FIG. 1. The learning data storage unit 80 may be the PACS server 58 described with reference to FIG. 2. The learning data storage unit 80 is an example of a “data storage apparatus of a medical institution” according to the present disclosure.

Here, an example in which the learning data storage unit 80 and the CAD server 60 that executes learning processing are configured as separate apparatuses will be described. However, the functions may be realized by one computer, or the processing functions may be shared and realized by two or more computers.

The computer-readable medium 604 of the CAD server 60 illustrated in FIG. 4 stores various programs, which include a local learning management program 630 and a diagnosis support program 640, and data. In a case where the processor 602 executes an instruction of the local learning management program 630, the computer functions as a synchronization processing unit 631, a learning data acquisition unit 632, a local model LM, an error calculation unit 634, an optimizer 635, a learning result storage unit 636, and a transmission processing unit 637. The local learning management program 630 is an example of a “second program” according to the present disclosure.

The synchronization processing unit 631 performs a communication with the integration server 30 via the communication interface 606, and synchronizes the master model MM in the integration server 30 with the local model LM in the CAD server 60.

The learning data acquisition unit 632 acquires learning data from the learning data storage unit 80. The learning data acquisition unit 632 may be configured to include a data input terminal for receiving data from an external apparatus or from another signal processing unit in the apparatus. Further, the learning data acquisition unit 632 may be configured to include a communication interface 606, an input/output interface 608, a media interface for performing reading and writing on a portable external storage medium such as a memory card (not illustrated), or an appropriate combination of these interfaces.

The learning data acquired via the learning data acquisition unit 632 is input to the local model LM as a learning model.

The error calculation unit 634 calculates an error between a predicted value indicated by a score which is output from the local model LM and the correct answer data. The error calculation unit 634 evaluates the error using a loss function. The loss function may be, for example, a cross entropy or a mean square error.

The optimizer 635 performs processing of updating a weight parameter of the local model LM from the calculation result of the error calculation unit 634. The optimizer 635 performs calculation processing of obtaining an update amount of the weight parameter of the local model LM and update processing of the weight parameter of the local model LM according to the calculated update amount of the weight parameter, by using the error calculation result obtained from the error calculation unit 634. The optimizer 635 updates the weight parameter based on an algorithm such as an error inverse propagation method.

The CAD server 60 in which the local learning management program 630 is incorporated functions as a local learning apparatus that executes machine learning on the CAD server 60 by using the local data LD as learning data. The CAD server 60 reads, from the learning data storage unit 80, the learning data as the local data LD, and executes machine learning. The CAD server 60 can read the learning data in units of mini-batch in which a plurality of pieces of learning data are collected, and update the weight parameter. A processing unit including the learning data acquisition unit 632, the local model LM, the error calculation unit 634, and the optimizer 635 is an example of a “learning processing unit” according to the present disclosure.

The local learning management program 630 repeats an iteration of learning processing until a learning end condition is satisfied. After the learning end condition is satisfied, the weight parameter of the local model LM is stored, as the learning result, in the learning result storage unit 636.

The transmission processing unit 637 performs processing of transmitting the learning result to the integration server 30. The weight parameter of the trained local model LM stored in the learning result storage unit 636 is transmitted to the integration server 30 via the communication interface 606 and the wide area communication line 70 (refer to FIG. 2). The transmission processing unit 637 and the communication interface 606 are examples of a “transmission unit” according to the present disclosure.

Further, in a case where the processor 602 executes an instruction of the diagnosis support program 640, the computer functions as an AI-CAD unit 642.

The AI-CAD unit 642 outputs an inference result for input data by using, as an inference model, the master model MINI or the local model LM. The input data to the AI-CAD unit 642 is, for example, a medical image such as a two-dimensional image, a three-dimensional image, and a moving image, and an output from the AI-CAD unit 642 is, for example, information indicating a position of a lesion portion in the image, information indicating a class classification such as a disease name, or a combination thereof.

<<Explanation of Local Learning Management Program 630>>

As described above, the local learning management program 630 is installed on the client terminal (client 20) existing in the medical institution network 50. Here, the client terminal may be, for example, the CAD server 60 in FIG. 2. The local learning management program 630 has a function of synchronizing the master model MINI with the local model LM before learning is performed, a function of starting local learning, a function of setting an end condition of local learning, and a function of transmitting the result of local learning to the integration server 30 when local learning is ended.

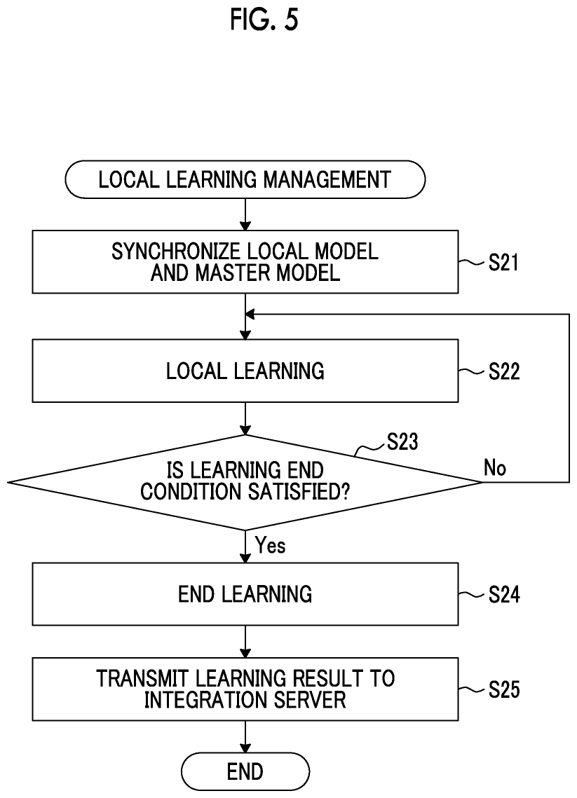

FIG. 5 is a flowchart illustrating an example of an operation of the client terminal based on the local learning management program 630. Steps in the flowchart illustrated in FIG. 5 are executed by the processor 602 according to an instruction of the local learning management program 630.

In step S21, at a time which is set by the local learning management program 630, the processor 602 of the CAD server 60 synchronizes the local model LM and the master model MM. Here, a “set time” may be designated as a fixed value, for example, a time outside of hospital examination business hours, or may be programmatically set by storing a record of an operating status of the CAD server 60 and determining a time when the CAD server 60 is not normally used.

In the synchronization of the local model LM and the master model MM, for example, a form in which a parameter file used by the model is updated and learning is performed by reading the parameter file via the program may be used, or a form in which the integration server 30 centrally manages a virtual container image and the terminal as the client 20 loads the virtual container image may be used. By the synchronization processing, the master model MINI is a learning model (local model LM) in an initial state before learning is started.

In step S22, the processor 602 executes local learning using the local data LD. The learning processing of the local model LM synchronized with the master model MINI is started up by the local learning management program 630, and local learning is performed with reference to the local data LD in the medical institution network 50.

In step S23, the processor 602 determines whether or not the learning end condition is satisfied. Here, the learning end condition includes, for example, the following conditions.

[Example 1] The number of iterations is designated in advance, and learning is ended after the designated number of iterations.

[Example 2] In a state where the verification data is stored in the medical institution network 50, an inference accuracy is calculated by performing accuracy comparison between an inference result obtained by inputting the verification data into the trained model and a correct answer, and learning is performed until accuracy improvement is achieved by the designated percentage. That is, the inference accuracy of the learning model is calculated using the verification data, and learning is ended in a case where accuracy improvement is achieved by the designated percentage.

[Example 3] A time limit is set, and learning is performed within the time limit. In a case where the time limit is reached, learning is ended.

An end condition of any one of [Example 1] to [Example 3] may be defined, or a logical product (AND) or a logical sum (OR) of a plurality of conditions may be set as an end condition.

In a case where a determination result in step S23 is a No determination, the processor 602 returns to step S22 and continues local learning processing. On the other hand, in a case where a determination result in step S23 is a Yes determination, the processor 602 proceeds to step S24, and learning is ended.

After learning is completed, in step S25, the processor 602 transmits the learning result to the integration server 30. For example, the processor 602 stores the trained model in a file format, and transmits the trained model to the integration server 30 via the wide area communication line 70.

Each of the plurality of the CAD servers 60 illustrated in FIG. 2 executes machine learning of each local model LM by using, as learning data, data stored in the PACS server 58 in different medical institution networks, and transmits a learning result to the integration server 30 via the wide area communication line 70.

<<Explanation of Learning Client Selection Program 38>>

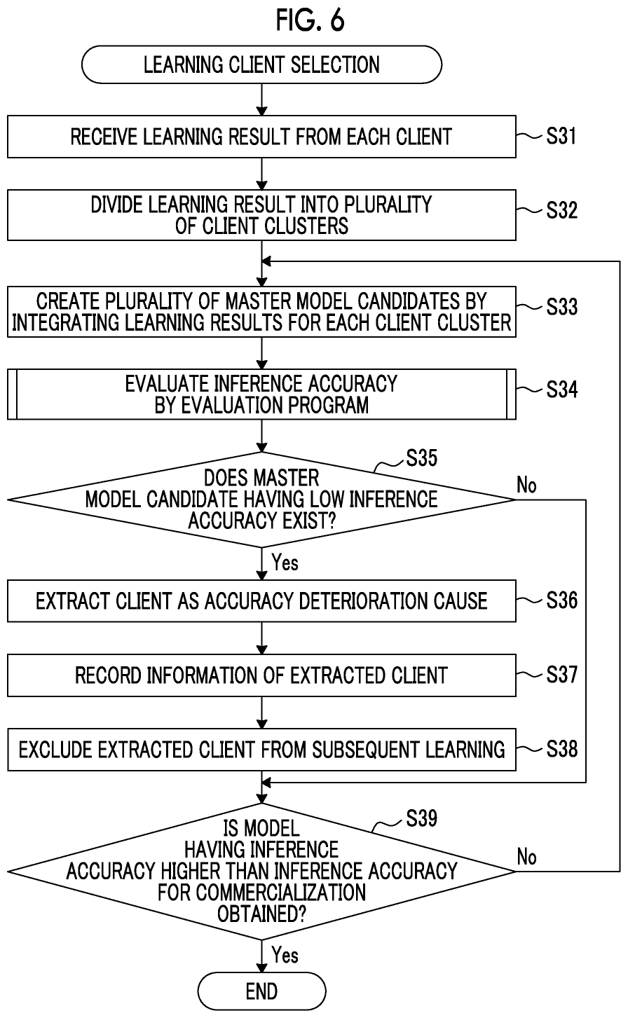

FIG. 6 is a flowchart illustrating an example of an operation of the integration server 30 based on the learning client selection program 38. Steps in the flowchart illustrated in FIG. 6 are executed by the processor 302 according to an instruction of the learning client selection program 38.

In step S31, the processor 302 receives the learning result from each client 20.

In step S32, the processor 302 divides the learning result transmitted from each client 20 into a plurality of client clusters. For example, the processor 302 randomly extracts the clients 20 from the population such that the number of the clients included in each client cluster is the same, and groups the plurality of clients 20.

In step S33, the processor 302 creates a plurality of master model candidates MMC by integrating the learning results for each client cluster. In a case where the master model candidate MMC is created by integrating the learning results of the client group and updating the weight parameter of the master model, as an integration method, a general federated learning algorithm may be used.

In a case of an existing federated learning method, a plurality of master model candidates are not created, and only a single master model exists. In this case, the single master model is updated by integrating a plurality of learning results of local learning. However, in a case of the existing method, it is difficult to exclude an influence of a client that causes an accuracy deterioration, and it is also difficult to specify a client as an accuracy deterioration cause.

On the other hand, in the present embodiment, a plurality of master model candidates MMC are created by dividing the learning results transmitted from each client 20 into a plurality of client groups (client clusters) and integrating the learning results for each client cluster.

Further, when creating the plurality of master model candidates MMC, the processor 302 stores, in the data storage unit such as the database 36, information indicating a correspondence relationship as to from which client cluster each master model candidate MMC is created.

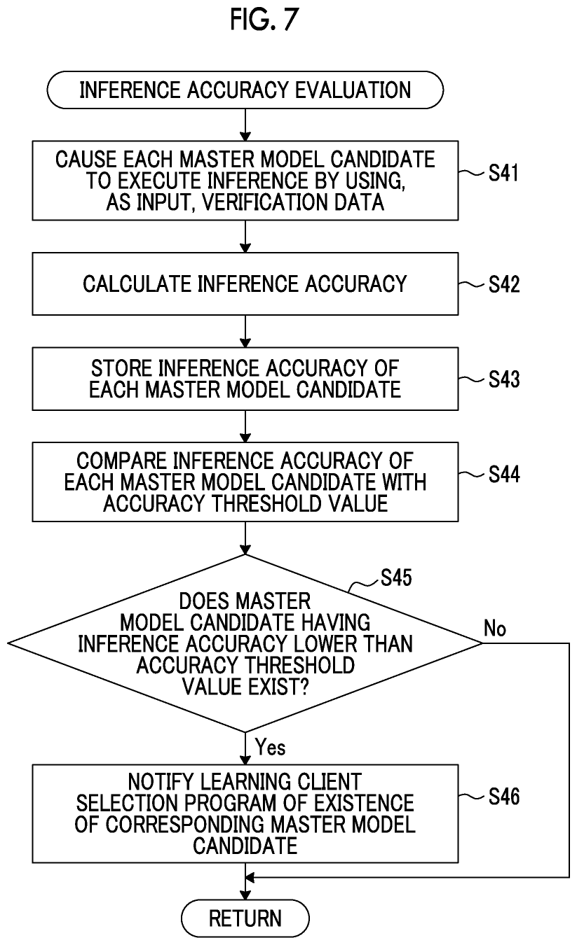

In step S34, the processor 302 evaluates the inference accuracy of each master model candidate MMC via the evaluation program 34. That is, the processor 302 causes each master model candidate MMC to perform an inference by using, as an input, the verification data TD existing in the integration server 30, calculates an inference accuracy, and stores the inference accuracy of each master model candidate MMC in the database 36. An example of processing contents via the evaluation program will be described later with reference to FIG. 7.

In step S35, the processor 302 determines whether or not a master model candidate MMC having an inference accuracy lower than the accuracy threshold value exists. The processor 302 can perform the determination in step S35 based on whether or not a notification for notifying of the existence of the master model candidate having an inference accuracy lower than the accuracy threshold value is received from the evaluation program.

In a case where a determination result in step S35 is a No determination, that is, in a case where a master model candidate MMC having an inference accuracy lower than the accuracy threshold value does not exist, the processor 302 does not change the relationship between each master model candidate MMC and the client cluster, and proceeds to step S39.

On the other hand, in a case where a determination result in step S35 is a Yes determination, that is, in a case where a master model candidate MMC having an inference accuracy lower than the accuracy threshold value exists, the processor 302 proceeds to step S36.

In step S36, the processor 302 extracts the client 20 as an accuracy deterioration cause of the master model candidate MMC having an inference accuracy lower than the accuracy threshold value. The processor 302 searches the client cluster for the master model candidate MMC having an inference accuracy lower than the accuracy threshold value, by using the information indicating the correspondence relationship between the master model candidate MMC stored in the database 36 by the processing of step S33 and the client cluster, and extracts the client as an accuracy deterioration cause.

Thereafter, in step S37, the processor 302 records, in the database 36, information of the client as an accuracy deterioration cause that is extracted by the processing of step S36.

In step S38, the processor 302 excludes the client as an accuracy deterioration cause that is extracted by the processing of step S36 from subsequent learning. After step S38, the processor 302 proceeds to step S39.

In step S39, the processor 302 determines whether or not a model having an inference accuracy higher than an inference accuracy for commercialization is obtained. In a case where a determination result in step S39 is a No determination, the processor 302 returns to step S33. That is, the processor 302 repeats processing of step S33 to step S39 until a model having an inference accuracy higher than an inference accuracy for commercialization is obtained. In a case where a determination result in step S39 is a Yes determination, the flowchart of FIG. 6 is ended.

<<Explanation of Evaluation Program 34>>

FIG. 7 is a flowchart illustrating an example of an operation of the integration server 30 based on the evaluation program 34. The flowchart illustrated in FIG. 7 is applied to step S34 of FIG. 6.

In step S41 of FIG. 7, the processor 302 causes each master model candidate MMC to execute an inference by using, as an input, the verification data TD.

In step S42, the processor 302 calculates an inference accuracy of each master model candidate MMC based on the inference result and the correct answer data.

In step S43, the processor 302 stores the inference accuracy of each master model candidate MMC in the database 36.

In step S44, the processor 302 compares the inference accuracy of each master model candidate MMC with the accuracy threshold value. Here, the accuracy threshold value may be compared with an instantaneous value of the inference accuracy of the master model candidate. However, in the comparison, while maintaining the configuration of the client cluster used for creation of each master model candidate MMC, a procedure of step S31 to step S43 may be performed for several iterations, the inference accuracy at that time may be recorded each time, and a statistical value such as an average value or a median value of the inference accuracy may be compared with the accuracy threshold value.

In step S45, the processor 302 determines whether or not a master model candidate MMC having an inference accuracy lower than the accuracy threshold value exists based on the comparison result in step S44.

In a case where a determination result in step S45 is a Yes determination, the processor 302 proceeds to step S46. In step S46, the processor 302 notifies the learning client selection program 38 of the existence of the master model candidate having an inference accuracy lower than the accuracy threshold value. Here, as a notification method, a message queue, a general inter-process communication, or the like may be used.

After step S46, or in a case where a determination result in step S45 is a No determination, the processor 302 ends the flowchart of FIG. 7 and returns to the flowchart of FIG. 6.

<<Specific Example of Processing by Integration Server 30>>

Here, a more specific example of processing by the integration server 30 will be described. The following processing of [Procedure 301] to [Procedure 311] is executed by the integration server 30 including the learning client selection program 38 and the evaluation program 34.

[Procedure 301] The learning client selection program 38 divides the learning client group client_id_array=[1, 2, 3, . . . , N, N+1] into K groups of the client clusters. For example, the learning client selection program 38 creates client_cluster__1=[1, 3, . . . , 2N+1] and client_cluster_2=[2, 4, . . . , 2].

[Procedure 302] The learning client selection program 38 creates K master model candidates MMC from the learning results of the K groups of client_cluster_*. “*” represents an index of the client cluster.

[Procedure 303] Thereafter, each master model candidate MMC is caused to perform an inference using the verification data TD, and a feedback on the obtained inference result from an evaluator is collected.

[Procedure 304] A feedback on the inference result of each master model candidate is collected. As a result, for example, in a case where a grade (inference accuracy) of client_cluster_X=[. . . , x, x+1, x+2, . . . ] is lower than the accuracy threshold value, an alert indicating this fact is transmitted from the evaluation program 34 to the learning client selection program 38.

[Procedure 305] In a case where the alert is received, the learning client selection program 38 divides client_cluster_X into L sub-clusters client_cluster_X_1, client_cluster_X_2, . . . , and client_cluster_X_L. L is an integer equal to or smaller than the number of the clients Q in the client cluster.

The number of the clients in each of the sub-clusters client_cluster_X_1, client_cluster_X_2, . . . , and client_cluster_X_L may be the same.

[Procedure 306] The learning client selection program 38 sets client cluster Ra by mixing client_cluster_X_1, . . . , and client_cluster_X_L into a part of the client cluster client cluster R, which has no problem in the inference accuracy in the previous learning. For example, the learning client selection program 38 sets client cluster 2a=client_cluster_2+client_cluster_X_i by mixing the sub-cluster client_cluster_X_i into the client cluster client cluster 2, which has no problem in the inference accuracy in the previous learning. Here, i represents an integer equal to or larger than 1 and equal to or smaller than L.

[Procedure 307] For client cluster Ra, as in procedure 301 to procedure 303, an inference model (master model candidate) is created, and a feedback on the inference accuracy is collected.

[Procedure 308] In a case where the inference accuracy of the model created from client cluster Ra is lower than the accuracy threshold value due to the mixing of client_cluster_X_i, it is determined that there is a high probability that client_cluster_X_i includes the client which causes a problem. In this case, the process proceeds to the next procedure 309, and the clients are narrowed down.

[Procedure 309] client_cluster_X_i is divided into p sub-clusters again, and procedure 305 to procedure 308 are repeated.

[Procedure 310] In this way, among the client cluster, the client cluster that is likely to have a problem, in which the number of the clients is sufficiently reduced, is set to client_cluster_low_prec_array as the accuracy deterioration cause. In subsequent learning, the clients in client_cluster_low_prec_array are not used. The number of the clients in client_cluster_low_prec_array may be equal to or larger than 1. A client other than the client as the accuracy deterioration cause may be included in client_cluster_low_prec_array.

[Procedure 311] As a master model to be used for a product, for example, from the master model candidates which remain after the process, that is, from the master model candidates having no problem in accuracy, the master model having a highest inference accuracy is selected and used.

In this way, the new master model created by performing the machine learning method using the machine learning system 10 according to the present embodiment is a master model having an improved inference accuracy as compared with the master model before learning is performed.

According to the present embodiment, it is possible to update an inference performance of the master model MM. In a case where the new master model created by performing the machine learning method according to the present embodiment is provided by sales or the like, preferably, the number of the clients used for learning, the number of pieces of verification data used for verification of the accuracy, and the like are described in an attached document provided in sales. For the number of the clients used for learning, as a client profile, for example, such as “hospital_how many cases”, “bed clinic_how many cases”, and “bedless clinic_how many cases”, preferably, a classification of the clients is displayed.

As a preliminary procedure in a case where a version of the master model as a current product is upgraded, information indicating the inference accuracy in the previous version and the inference accuracy in the new version and information indicating the number of the clients used for additional learning and the classification of the clients are presented to a medical institution, and an approval is received from the medical institution before the version is upgraded. After an approval is obtained, the version is upgraded.

<<Example of Hardware Configuration of Computer>>

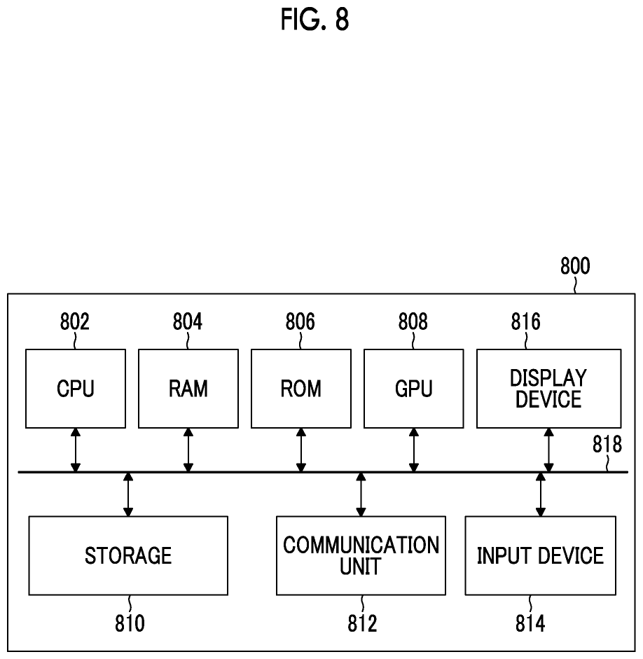

FIG. 8 is a block diagram illustrating an example of a hardware configuration of a computer. A computer 800 may be a personal computer, a workstation, or a server computer. The computer 800 may be used as a part or all of the client 20, the integration server 30, the PACS server 58, the CAD server 60, and the terminal 62 described above, or may be used as an apparatus having a plurality of functions thereof.

The computer 800 includes a CPU 802, a random access memory (RAM) 804, a read only memory (ROM) 806, a GPU 808, a storage 810, a communication unit 812, an input device 814, a display device 816, and a bus 818. The GPU 808 may be provided as necessary.

The CPU 802 reads out various programs stored in the ROM 806, the storage 810, or the like, and executes various processing. The RAM 804 is used as a work area of the CPU 802. Further, the RAM 804 is used as a storage unit for temporarily storing the read program and various data.

The storage 810 includes, for example, a hard disk device, an optical disk, a magneto-optical disk, a semiconductor memory, or a storage device configured by using an appropriate combination thereof. The storage 810 stores various programs, data, and the like required for inference processing and/or learning processing. The program stored in the storage 810 is loaded into the RAM 804, and the CPU 802 executes the program. Thus, the computer 800 functions as means for performing various processing defined by the program.

The communication unit 812 is an interface that performs communication processing with an external apparatus in a wired manner or a wireless manner and exchanges information with the external apparatus. The communication unit 812 may play a role of an information acquisition unit that receives an input such as an image.

The input device 814 is an input interface that receives various operation inputs to the computer 800. The input device 814 is configured with, for example, a keyboard, a mouse, a touch panel, another pointing device, a voice input device, or an appropriate combination thereof.

The display device 816 is an output interface for displaying various information. The display device 816 is configured with, for example, a liquid crystal display, an organic electro-luminescence (OEL) display, a projector, or an appropriate combination thereof.

<<Program for Operating Computer>>

A program causing a computer to realize a part or all of at least one processing function among various processing functions described in the embodiment may be recorded on a computer-readable medium as a non-transitory tangible information storage medium such as an optical disk, a magnetic disk, or a semiconductor memory, and the program may be provided via the information storage medium, the various processing functions including a local learning function of each client 20, a learning client selection function including a master model candidate creation function and an inference accuracy evaluation function of the integration server 30, and the like.

Further, instead of the form in which the program is provided by being stored in a non-transitory tangible computer-readable medium, a program signal may be provided as a download service using a telecommunication line such as the Internet.

Further, a part or all of at least one processing function among a plurality of processing functions including the local learning function, the learning client selection function, and the inference accuracy evaluation function described in the embodiment may be provided as an application server, and a service for providing the processing function via a telecommunication line may be performed.

<<Hardware Configuration of Each Processing Unit>>

As a hardware structure of the processing unit that executes various processing, such as the master model storage unit 320, the verification data storage unit 322, the client cluster extraction unit 332, the master model candidate creation unit 334, the accuracy deterioration cause extraction unit 336, the exclusion processing unit 338, the inference unit 342, the inference accuracy calculation unit 344, the accuracy threshold value comparison unit 346, the display control unit 350, which are illustrated in FIG. 3, the synchronization processing unit 631, the learning data acquisition unit 632, the local model LM, the error calculation unit 634, the optimizer 635, the learning result storage unit 636, the transmission processing unit 637, the AI-CAD unit 642, and the display control unit 650, which are illustrated in FIG. 4, for example, the following various processors may be used.

The various processors include a CPU which is a general-purpose processor that functions as various processing units by executing a program, a GPU which is a processor specialized for image processing, a programmable logic device (PLD) such as a field programmable gate array (FPGA) which is a processor capable of changing a circuit configuration after manufacture, a dedicated electric circuit such as an application specific integrated circuit (ASIC) which is a processor having a circuit configuration specifically designed to execute specific processing, and the like.

One processing unit may be configured by one of these various processors, or may be configured by two or more processors having the same type or different types. For example, one processing unit may be configured by a plurality of FPGAs, a combination of a CPU and an FPGA, or a combination of a CPU and a GPU. Further, the plurality of processing units may be configured by one processor. As an example in which the plurality of processing units are configured by one processor, firstly, as represented by a computer such as a client and a server, a form in which one processor is configured by a combination of one or more CPUs and software and the processor functions as the plurality of processing units may be adopted. Secondly, as represented by a system on chip (SoC) or the like, a form in which a processor that realizes the function of the entire system including the plurality of processing units by one integrated circuit (IC) chip is used may be adopted. As described above, the various processing units are configured by using one or more various processors as a hardware structure.

Further, as the hardware structure of the various processors, more specifically, an electric circuit (circuitry) in which circuit elements such as semiconductor elements are combined may be used.

### Advantages according to Present Embodiment

According to the machine learning system 10 according to the embodiment of the present invention, the following advantages are obtained.

[1] Learning can be performed without extracting personal information such as a diagnosis image that requires consideration for privacy from a medical institution.

[2] The client that is not suitable for learning and that is an accuracy deterioration cause can be extracted from the plurality of clients 20, and thus, the client having a problem can be excluded from the learning client group.

[3] It is possible to ensure the learning accuracy in federated learning, and it is possible to prevent an accuracy decrease of the master model caused by some clients having a problem.

[4] It is possible to create an AI model having a high inference accuracy.

### Modification Example 1

In the embodiment, the AI model for medical image diagnosis has been described as an example. However, the scope of application of the technique of the present disclosure is not limited to this example. For example, the present disclosure may be applied even in a case where learning is performed on an AI model using time-series data as input data or an AI model using document data as input data. The time-series data may be, for example, electrocardiogram waveform data. The document data may be, for example, a diagnosis report, and the present disclosure may be applied to training of an AI model for supporting creation of a report.

### Modification Example 2

In addition to or instead of measures to exclude the client as the accuracy deterioration cause from subsequent learning, the integration server 30 may notify the client as the accuracy deterioration cause of a fact that there is a problem.

<<Other>>

The matters described in the configuration and the modification example described in the embodiment may be used in combination as appropriate, and some matters may be replaced. The present invention is not limited to the embodiment described above, and various modifications may be made without departing from the scope of the present invention.

### EXPLANATION OF REFERENCES

10: machine learning system
20: client
30: integration server
34: evaluation program
36: database
38: learning client selection program
50: medical institution network
52: CT apparatus
54: MM apparatus
56: CR apparatus
58: PACS server
60: CAD server
62: terminal
64: internal communication line
70: wide area communication line
80: learning data storage unit
302: processor
304: computer-readable medium
306: communication interface
308: input/output interface
310: bus
314: input device
316: display device
320: master model storage unit
322: verification data storage unit
324: synchronization program
332: client cluster extraction unit
334: master model candidate creation unit
336: accuracy deterioration cause extraction unit
338: exclusion processing unit
342: inference unit
344: inference accuracy calculation unit
346: accuracy threshold value comparison unit
350: display control unit
602: processor
604: computer-readable medium
606: communication interface
608: input/output interface
610: bus
614: input device
616: display device
630: local learning management program
631: synchronization processing unit
632: learning data acquisition unit
634: error calculation unit
635: optimizer
636: learning result storage unit
637: transmission processing unit
640: diagnosis support program
642: AI-CAD unit
650: display control unit
800: computer
802: CPU
804: RAM
806: ROM
808: GPU
810: storage
812: communication unit
814: input device
816: display device
818: bus
DM: devil mark
LD: local data
LM: local model
MM: master model
MMC: master model candidate
MMC1: master model candidate
MMCK: master model candidate
TD: verification data
S21 to S25: steps of local learning management processing
S31 to S39: steps of learning client selection processing
S41 to S46: steps of inference accuracy evaluation processing

What is claimed is:

1. A machine learning system comprising:
a plurality of client terminals; and
an integration server,
wherein the integration server comprises
a first processor and
a non-transitory first computer-readable medium storing a trained master model, each of the plurality of client terminals comprises
a second processor,
the second processor is configured to:
execute machine learning of a learning model using, as learning data, data stored in a data storage apparatus of a medical institution; and
transmit a learning result of the learning model to the integration server, and the first processor is configured to:
synchronize the learning model of each client terminal with the master model before training of the learning model is performed on each of the plurality of client terminals;
receive each of the learning results from the plurality of client terminals;
create a plurality of client clusters by dividing the plurality of client terminals into a plurality of groups;
create master model candidates for each of the client clusters by integrating the learning results for each of the client clusters;
detect the master model candidate having an inference accuracy lower than an accuracy threshold value by evaluating the inference accuracy of each of the master model candidates created for each of the client clusters; and
extract a client terminal as an accuracy deterioration cause from the client cluster used for creation of the master model candidate having the inference accuracy lower than the accuracy threshold value.

2. The machine learning system according to claim 1,
wherein the first processor is configured to exclude the client terminal as the accuracy deterioration cause from subsequent learning.

3. The machine learning system according to claim 1,
wherein the first computer-readable medium stores information of the client terminal as the accuracy deterioration cause.

4. The machine learning system according to claim 1,
wherein each of the plurality of client terminals is a terminal provided in a medical institution network of different medical institutions.

5. The machine learning system according to claim 1,
wherein the integration server is provided in a medical institution network or outside the medical institution network.

6. The machine learning system according to claim 1,
wherein the learning result transmitted from the client terminal to the integration server includes a weight parameter of the trained learning model.

7. The machine learning system according to claim 1,
wherein the data used as the learning data includes at least one type of data among a two-dimensional image, a three-dimensional image, a moving image, time-series data, and document data.

8. The machine learning system according to claim 1,
wherein each model of the learning model, the master model, and the master model candidate is configured by using a neural network.

9. The machine learning system according to claim 1,
wherein the data used as the learning data includes a two-dimensional image, a three-dimensional image, or a moving image, and
each model of the learning model, the master model, and the master model candidate is configured by using a convolutional neural network.

10. The machine learning system according to claim 1,
wherein the data used as the learning data includes time-series data or document data, and
each model of the learning model, the master model, and the master model candidate is configured by using a recursive neural network.

11. The machine learning system according to claim 1,
wherein the number of the client terminals included in each of the plurality of client clusters is the same, and the client terminal included in each client cluster is not overlapped.

12. The machine learning system according to claim 1,
wherein the first computer-readable medium stores information indicating a correspondence relationship as to which client cluster among the plurality of client clusters each of the plurality of master model candidates created is based on.

13. The machine learning system according to claim 1,
wherein the first processor is configured to determine whether or not the inference accuracy of the master model candidate is lower than the accuracy threshold value based on a comparison between an instantaneous value of the inference accuracy of each of the master model candidates and the accuracy threshold value, or based on a comparison between a statistical value of the inference accuracy in a learning iteration of each of the master model candidates and the accuracy threshold value.

14. The machine learning system according to claim 1,
wherein, in a case where the master model candidate having the inference accuracy lower than the accuracy threshold value is detected, the first processor is configured to notify the accuracy deterioration cause extraction unit of information of the master model candidate related to the detection.

15. The machine learning system according to claim 1,
wherein the integration server further comprises a display device,
the display device is configured to display the inference accuracy in each learning iteration of each of the master model candidates created for each of the client clusters.

16. The machine learning system according to claim 1, further comprising:
a verification data storage that stores verification data,
wherein the first processor is configured to evaluate the inference accuracy of the master model candidate using the verification data.

17. A machine learning method using a plurality of client terminals and an integration server, the method comprising:
synchronizing a learning model of each client terminal with a trained master model stored in the integration server before training of the learning model is performed on each of the plurality of client terminals;
executing machine learning of the learning model using, as learning data, data stored in a data storage apparatus of each of medical institutions different from each other by each of the plurality of client terminals;
transmitting a learning result of the learning model to the integration server from each of the plurality of client terminals;
receiving each of the learning results from the plurality of client terminals by the integration server;
creating a plurality of client clusters by the integration server, by dividing the plurality of client terminals into a plurality of groups;
creating master model candidates for each of the client clusters by the integration server, by integrating the learning results for each of the client clusters;
detecting the master model candidate having an inference accuracy lower than an accuracy threshold value by the integration server, by evaluating the inference accuracy of each of the master model candidates created for each of the client clusters; and
extracting a client terminal as an accuracy deterioration cause from the client cluster used for creation of the master model candidate having the inference accuracy lower than the accuracy threshold value by the integration server.

18. An integration server connected to a plurality of client terminals via a communication line, the server comprising:
a first processor; and
a first computer-readable medium as a non-transitory tangible medium in which a first program to be executed by the first processor is recorded,
wherein the first processor is configured to, according to an instruction of the first program,
store a trained master model on the first computer-readable medium,
synchronize a learning model of each client terminal with the master model before training of the learning model is performed on each of the plurality of client terminals,
receive each of learning results from the plurality of client terminals,
create a plurality of client clusters by dividing the plurality of client terminals into a plurality of groups,
create master model candidates for each of the client clusters by integrating the learning results for each of the client clusters,
detect the master model candidate having an inference accuracy lower than an accuracy threshold value by evaluating the inference accuracy of each of the master model candidates created for each of the client clusters, and
extract a client terminal as an accuracy deterioration cause from the client cluster used for creation of the master model candidate having the inference accuracy lower than the accuracy threshold value.

19. An information processing apparatus that is used as one of the plurality of client terminals connected to the integration server according to claim 18 via a communication line, the apparatus comprising:
a second processor; and
a second computer-readable medium as a non-transitory tangible medium in which a second program to be executed by the second processor is recorded,
wherein the second processor is configured to, according to an instruction of the second program,
execute machine learning of a learning model by setting, as the learning model in an initial state before learning is started, a learning model synchronized with the master model stored in the integration server and using, as learning data, data stored in a data storage apparatus of a medical institution, and
transmit a learning result of the learning model to the integration server.

20. A non-transitory computer readable medium storing a program causing a computer to function as one of the plurality of client terminals connected to the integration server according to claim 18 via a communication line, the program causing the computer to realize:
a function of executing machine learning of a learning model by setting, as the learning model in an initial state before learning is started, a learning model synchronized with the master model stored in the integration server and using, as learning data, data stored in a data storage apparatus of a medical institution; and
a function of transmitting a learning result of the learning model to the integration server.

21. A non-transitory computer readable medium storing a program causing a computer to function as an integration server connected to a plurality of client terminals via a communication line, the program causing the computer to realize:
a function of storing a trained master model;
a function of synchronizing a learning model of each client terminal with the master model before training of the learning model is performed on each of the plurality of client terminals;
a function of receiving each of learning results from the plurality of client terminals;
a function of creating a plurality of client clusters by dividing the plurality of client terminals into a plurality of groups;
a function of creating master model candidates for each of the client clusters by integrating the learning results for each of the client clusters;
a function of detecting the master model candidate having an inference accuracy lower than an accuracy threshold value by evaluating the inference accuracy of each of the master model candidates created for each of the client clusters; and
a function of extracting a client terminal as an accuracy deterioration cause from the client cluster used for creation of the master model candidate having the inference accuracy lower than the accuracy threshold value.

22. An inference model creation method for creating an inference model by performing machine learning using a plurality of client terminals and an integration server, the method comprising:
synchronizing a learning model of each client terminal with a trained master model stored in the integration server before training of the learning model is performed on each of the plurality of client terminals;
executing machine learning of the learning model using, as learning data, data stored in a data storage apparatus of each of medical institutions different from each other via each of the plurality of client terminals;
transmitting a learning result of the learning model to the integration server from each of the plurality of client terminals;
receiving each of the learning results from the plurality of client terminals by the integration server;
creating a plurality of client clusters by the integration server, by dividing the plurality of client terminals into a plurality of groups;
creating master model candidates for each of the client clusters by the integration server, by integrating the learning results for each of the client clusters;
detecting the master model candidate having an inference accuracy lower than an accuracy threshold value by the integration server, by evaluating the inference accuracy of each of the master model candidates created for each of the client clusters;
extracting a client terminal as an accuracy deterioration cause from the client cluster used for creation of the master model candidate having the inference accuracy lower than the accuracy threshold value by the integration server; and
creating the inference model having an inference accuracy higher than the inference accuracy of the master model based on the master model candidate having an inference accuracy equal to or higher than the accuracy threshold value by the integration server.
